(19) United States
(12) Patent Application Publication
(10) Pub. No.: US 2008/0161266 A1
(43) Pub. Date: Jul. 3, 2008

(54) Ophthalmic Alginate Composition Related Methods of Manufacture and Methods of Use

(76) Inventors: Dharmendra M. Jani, Fairport, NY (US); Stephen E. Maier, Brockport, NY (US)

Correspondence Address: Bausch & Lomb Incorporated, One Bausch & Lomb Place, Rochester, NY, 14604-2701, US

(21) Appl. No.: 11/961,447

(22) Filed: Dec. 20, 2007

Related U.S. Application Data
(60) Provisional application No. 60/882,655, filed on Dec. 29, 2006.

(51) Int. Cl. A61K 31/717; A61P 27/02; A61K 47/36; A61K 31/155; A61K 47/10

(52) U.S. Cl. 514/57; 514/779; 514/635; 514/772

(57) ABSTRACT

The present invention is directed to an ophthalmic composition comprising alginate having a minimum of about 0% and a maximum of about 50% guluronic units bound to an adjacent guluronic unit as a percentage of the total number of monomeric units in the alginate. The composition further includes a cationic antimicrobial agent. The alginate set forth above forms less deactivating complexes with cationic antimicrobial agent, and thus, the composition has improved preservative efficacy.

### CROSS REFERENCE

This application claims the benefit of Provisional Patent Application No. 60/882,655 filed Dec. 29, 2006, which is incorporated by reference herein.

### FIELD OF THE INVENTION

This invention relates to ophthalmic compositions comprising alginate, methods of manufacture, and methods of use of such compositions.

### BACKGROUND

Ophthalmic compositions are used for several purposes including the relief and treatment of ophthalmic disease, cleaning, conditioning and disinfection of contact lenses and treatment of dry eye. Whether treating dry eye, delivering an active pharmaceutical agent to the ocular tissue, or cleaning or conditioning contact lenses, polymeric demulcents are useful. For delivery of active pharmaceutical agents, polymeric demulcents increase the residence time in the eye of an active pharmaceutical agent. Polymeric demulcents retain moisture in the ocular tissue to relieve dry eye discomfort. Overall comfort can be improved by the use of polymeric demulcents in contact lens cleaning, disinfecting, conditioning or multipurpose solutions.

Ophthalmic solutions that are used and reused are needed to provide long-term preservative efficacy in the case of ophthalmic compositions for delivery of active pharmaceutical agents, dry eye treatments, and contact lens conditioning, cleaning or rewetting drops. Ophthalmic solutions that disinfect contact lenses, including multipurpose contact lens solutions, require long-term stable disinfecting properties. It is known that a potential exists for ingredients other than antimicrobial agents to increase or decrease the antimicrobial efficacy of the antimicrobial agents. Any ingredient that can increase the antimicrobial efficacy without compromising the comfort or increasing toxicity is valuable. Any ingredient that can minimize the complexation between a demulcent and an antimicrobial agent is likewise advantageous. Alginate is a polysaccharide that is known for use as a demulcent in ophthalmic solutions. Alginate, for the purpose of this application, is a polysaccharide that comprises β-D-mannuronic acid and α-L-guluronic acid monomers or salts or derivatives of such acids or salts.

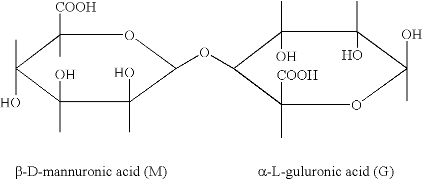

Some alginate polymers are block copolymers with blocks of the guluronic acid (or salt) monomers alternating with blocks of the mannuronic acid (or salt) monomers. Some alginate molecules have single monomers of guluronic acid (or salt) alternating with the comonomers of mannuronic acid (or salt). The ratio and distribution of the M and G components, along with the average molecular weight, affect the physical and chemical properties of the copolymer. See Haug, A. et al., Acta Chem. Scand., Vol., 183-90 (1966). Alginate polymers have viscoelastic Theological properties and other properties that make it suitable for some medical applications. See Klock, G. et al., “Biocompatibility of mannuronic acid-rich alginates,” Biomaterials, Vol. 18, No. 10, 707-13 (1997).

The use of alginate as a thickener for topical ophthalmic use is disclosed in U.S. Pat. No. 6,528,465 and U.S. Patent Application Publication 2003/0232089 incorporated herein by reference in their entirety. In U.S. Pat. No. 5,776,445, alginate is used as a drug delivery agent that is topically applied to the eye. Particularly, the amount of guluronic acid in the alginate was taught to exceed 50%.

U.S. Patent Publication No. 2003/0232089 teaches a dry-eye formulation that contains two polymer ingredients including alginate.

Polyols including glycerin are known as demulcents and tonicity adjusting agents in ophthalmic formulations including formulations for the delivery of an active pharmaceutical agent. See U.S. Pat. Nos. 5,075,104 and 5,209,927 which teach the use of a polyol with a cabomer polymer.

In view of the above, it would be desirable to provide an ophthalmic solution that contains an effective polymeric demulcent that increases the residence time in the eye of active pharmaceutical agents, improves long-lasting wetting properties, improves comfort while minimizing the amount of complexation with antimicrobial agents. The present invention addresses these and other needs.

### SUMMARY OF THE INVENTION

The present invention is an ophthalmic composition comprising an aqueous composition of alginate having a minimum of about 0% and a maximum of about 50% guluronic units bound to an adjacent guluronic unit as a percentage of the total number of monomeric units in the alginate composition. The composition comprises a cationic antimicrobial agent. In one embodiment, the ophthalmic solution is for treatment of a dry eye condition. Such a composition has improved coating properties, remains in the eye for a longer period of time, and can relieve symptoms of dry eye. There is a reduced interaction between the alginate and the cationic antimicrobial agent. A strong interaction between alginate and an antimicrobial agent negatively impacts the efficacy of the antimicrobial agent. A composition of the present invention avoids such strong interaction and, thus, offers superior efficacy in antimicrobial activity.

In another embodiment, the ophthalmic composition is an ophthalmic composition for treatment of ocular disease and contains an active pharmaceutical agent. In another embodiment, the solution is a contact lens treatment solution, such as a contact lens conditioning solution, a contact lens cleaning solution, a contact lens disinfecting solution, or a solution that accomplishes one or more of the above such as a multipurpose cleaning solution.

The present invention also comprises, in one aspect, a method of treating dry eye. The method comprises administering to an eye a composition comprising an aqueous solution of alginate having a minimum of about 0% and a maximum of about 50% guluronic units bound to an adjacent guluronic unit as a percentage of the total number of monomeric units in the alginate polysaccharide. The composition also comprises a cationic antimicrobial agent.

In still another embodiment, there is a method for manufacturing an ophthalmic composition. The method comprises combining, in an aqueous solution, ophthalmically pure alginate having a minimum of about 0% and a maximum of about 50% guluronic units bound to an adjacent guluronic unit as a percentage of the total number of monomeric units in the alginate polysaccharide. The composition also comprises a cationic antimicrobial agent.

### DETAILED DESCRIPTION OF THE INVENTION

The present invention is directed to a dry eye composition comprising an aqueous solution of alginate having a minimum of about 0% and a maximum of about 50% guluronic units bound to an adjacent guluronic unit as a percentage of the total number of monomeric units in the alginate polysaccharide. The composition has been shown to form less complexes between the alginate and cationic antimicrobial agent. This minimizes the decrease in antimicrobial efficacy that may result from such complexation. The ophthalmic solution is a dry eye treatment solution in one embodiment. In another embodiment, the ophthalmic solution is a vehicle for delivering an active pharmaceutical. In still another embodiment, the solution is a contact lens care cleaning, conditioning and disinfecting solution.

In one embodiment, the alginate has a minimum of about 0%, about 5%, about 7% or about 10% and/or a maximum of about 50%, about 40%, about 30%, about 20%, about 15% guluronic units bound to adjacent guluronic units based upon the total number of monomeric units. Preferably, the alginate has a minimum of about 60%, and a maximum of about 70% guluronic units bound to adjacent guluronic units based upon the total number of monomeric units in the alginate polysaccharide.

In one embodiment, the alginate has a minimum of about 50%, about 60%, about 70% or about 80% and/or a maximum of about 100%, about 90%, about 80%, about 70% or about 65% mannuronic units bound to adjacent mannuronic units or guluronic units based upon the total number of monomeric units in the alginate polysaccharide. Preferably, the alginate has a minimum of about 70%, and a maximum of about 80% mannuronic units bound to adjacent mannuronic units or guluronic units based upon the total number of monomeric units in the alginate polysaccharide.

The alginate of one embodiment has a molecular weight that is a minimum of about 1 kDa, about 20 kDa, about 50 kDa, about 80 kDa, about 100 kDa, about 500 kDa and/or a maximum of about 5000 kDa, about 2000 kDa, about 1000 kDa, about 700 kDa, about 500 kDa, about 200 kDa or about 100 kDa. In one preferred embodiment, the molecular weight is about 325 kDa.

The concentration of alginate is a minimum of about 0.01 wt. % and a maximum of about 2.0 wt. % based upon the total weight of the solution. Typically, the concentration of alginate is a minimum of about 0.05 wt. %, about 0.1 wt. %, about 0.25%, about 0.5 wt. % or about 1 wt. % based upon the total weight of the solution. Typically, the concentration of alginate is a maximum of about 5 wt. %, about 3 wt. %, about 2 wt. %, about 1.5 wt. % and about 1.2 wt. % based upon the total weight of the solution. Preferably, the concentration of alginate is about 0.5 wt. % based upon the total weight of the solution.

The present composition may contain a cationic antimicrobial agent in a disinfecting amount or a preserving amount. Antimicrobial agents are defined as organic chemicals that derive their antimicrobial activity through a chemical or physiochemical interaction with the microbial organisms. Cationic antimicrobial agents are antimicrobial agents that have a positive charge in solution. These include quarternary ammonium compounds (including small molecules) and polymers and low and high molecular weight biguanides. For example, biguanides include the free bases or salts of Alexidine, chlorhexidine, hexamethylene biguanides and their polymers, and combinations of the foregoing. The salts of Alexidine and chlorhexidine can be either organic or inorganic. They are typically gluconates, nitrates, acetates, phosphates, sulfates, halides and the like.

A preferred polymeric biguanide is poly(hexamethylene biguanide) (PHMB) commercially available from Zeneca, Wilmington, Del. under the trademark Cosmocil™ CQ. Generally, the hexamethylene biguanide polymers, also referred to as poly(aminopropyl biguanide) (PAPB), have molecular weights of up to about 100 kDa. A particularly preferred preservative is Alexidine. Other cationic antimicrobial agents includes polyquatemium-1, polyquaternium-10, quaternary ammonium salts of chitosan, quaternary ammonium salts of guar (including guar hydroxypropyltrimonium chloride), and other quaternary ammonium derivatives of polysaccharides.

If used in the subject solution, the antimicrobial agent should be used in an amount which will preserve or prevent the growth of the microorganism population in the formulations employed. Preferably, a preservative amount is that which will reduce the bacterial bioburden after 28 days each by 3 logs and prevents the growth of fungal bioburden. Typically, such agents are present in a minimum concentration of about 0.0001 wt. %, 0.0003 wt. % or 0.0005 wt. % and/or a maximum concentration of about 0.0005 wt. %, about 0.001 wt. % or about 0.005 wt. % based upon the total weight of the composition.

In one embodiment, the formulation comprises a polyol. The polyol of the present invention is typically a polyol containing 2 to 6 carbon atoms. Preferably, the polyol contains 2 to 4 carbon atoms. The polyol, of one embodiment, is selected from the group consisting of glycerin, ethylene glycol, poly(ethylene glycol), propylene glycol, sorbitol, manitol and monosaccarides, disaccharides, oligosaccharides and neutral polysaccharide. In one preferred embodiment, the polyol is selected from the group consisting of glycerin, ethylene glycol, propylene glycol, sorbitol, mannitol and monosaccharides. In another preferred embodiment, the polyol is selected from the group comprising disaccharides, oligosaccharides and poly(ethylene glycol). In one preferred embodiment, the polyol is glycerin.

The concentration of polyol including glycerin is a minimum of about 0.01 wt. %, about 0.05 wt. %, about 0.1 wt. %, about 0.5 wt. % or about 1.0 wt. %, and/or a maximum of about 1.5 wt. %, about 2.0 wt. %, about 3.0 wt. %, about 4.0 wt. % or about 5 wt. % based upon the total weight of the composition.

In one embodiment the polyol is a combination of glycerin and propylene glycol. Typically, the ratio of glycerin to propylene glycol is a minimum of about 30:70, about 35:65, about 40:60 or about 45:55. The ratio of glycerin to propylene glycol is a maximum of about 70:30, about 65:35, about 60:40 or about 55:45. In one embodiment, the ratio of glycerin to propylene glycol is 1:1. In one embodiment, the concentration of glycerin is a minimum of about 0.1 wt. %, about 0.3 wt. %, about 0.4 wt. % or about 0.5 wt. % and/or a maximum of about 0.8 wt. %, about 0.9 wt. %, about 1 wt. %, about 1.5 wt. %, about 2 wt. % or about 3 wt. % based upon the total weight of the composition. In one embodiment the concentration of propylene glycol is a minimum of about 0.1 wt. %, about 0.3 wt. %, about 0.4 wt. % or about 0.5 wt. % and/or a maximum of about 0.8 wt. %, about 0.9 wt. %, about 1 wt. %, about 1.5 wt. %, about 2 wt. % or about 3 wt. % based upon the total weight of the composition.

According to one embodiment, the ratio of alginate to polyol is a minimum of about 1:20, about 1:4, about 1:3, about 1:2, about 2:3 or about 3:4 and/or a maximum of about 20:1, about 4:1, about 3:1, about 2:1, about 3:2 or about 4:3.

The aqueous solutions employed in this invention may contain additional ingredients to those described above. One or more other components that are commonly present in ophthalmic solutions, for example, buffers, stabilizers, tonicity agents and the like aid in making ophthalmic compositions more comfortable to the user.

The aqueous solutions of the present invention are typically adjusted with tonicity agents to approximate the tonicity of normal lacrimal fluids which is equivalent to a 0.9 wt. % solution of sodium chloride or a 2.8 wt. % of glycerol solution. The solutions are made substantially isotonic with physiological saline used alone or in combination. Correspondingly, excess salt or other tonicity agents may result in the formation of a hypertonic solution that will cause stinging and eye irritation. The osmolality of the composition of one embodiment is a minimum of about 200 mOsm/kg, about 225 mOsm/kg, about 250 mOsm/kg, about 260 mOsm/kg, about 280 mOsm/kg, about 300 mOsm/kg or about 320 mOsm/kg and/or a maximum of about 400 mOsm/kg, about 380 mOsm/kg, about 360 mOsm/kg, about 340 mOsm/kg or about 320 mOsm/kg. Most preferably, the osmolality is from about 240 mOsm/kg to about 320 mOsm/kg.

Preferably, the composition of at least one embodiment of the present invention has a low ionic strength. Typically, the composition contains low concentration of mono or divalent cations typically found in tear fluids. Generally, the composition contains a low concentration of one or more of the following cations: Na+, K+, Ca++, Mg++, and Zn++. In one embodiment, the concentration of the mono or divalent cations that are typically found in tear fluids (eg, Na+, K+, Ca++, Mg++ and Zn++) has a minimum concentration of about 0.001 wt. %, about 0.005 wt. % or about 0.01 wt. % and/or a maximum of about 0.1 wt. %, about 0.01 wt. %, about 0.1 wt. %, or about 0.05 wt. % based upon the total weight of the composition.

The pH of the composition of one embodiment is used to treat dry eye it should be maintained at a minimum of about 4, about 5, about 5.5, about 6, about 6.5 and/or a maximum of about 7.5, about 7.8, about 8, about 8.5. Suitable buffers may be added, such as borate, citrate, bicarbonate, aminoalcohol buffers, MOPS buffer, bicine, tricine, TRIS, BIS/TRIS and various mixed phosphate buffers (including combinations of Na₂HPO₄, NaH₂PO₄ and KH₂PO₄) and mixtures thereof. Preferred combination buffers include borate/phosphate and borate/citrate combination buffers. Generally, buffers will be used in amounts having a minimum of about 0.05 wt. % or about 0.1 wt. % and/or a maximum of about 1.5 wt. % or about 2.5 wt. %.

In some instances it may be desirable to include sequestering agents in the present solutions in order to bind metal ions, which might otherwise react with the components in the solution, the contact lens and/or protein deposits on a contact lens. Ethylene-diaminetetraacetic acid (EDTA) and its salts (disodium) and hydroxyalkylphosphonate are preferred examples. Typically, the sequestering agents are added in amounts having a minimum of about 0.01 wt. % and/or a maximum of about 0.5 wt. %.

The present invention includes a method of treating dry eye comprising administering to an eye a composition comprising an aqueous solution of alginate and a cationic antimicrobial agent. The alginate has a minimum of 0% and a maximum of 50% guluronic acid monomers bound to an adjacent guluronic acid monomer based upon the total number of monomers in the alginate polysaccharide. The method further includes administering to an eye a composition according to any one or more embodiments or combination of embodiments disclosed herein.

In one embodiment, there is a method of manufacturing an ophthalmic composition. The method of manufacturing comprises combining in an aqueous solution ophthalmically pure alginate (eg. sodium alginate) having a minimum of about 0% and a maximum of about 50% guluronic units bound to an adjacent guluronic unit as a percentage of the total number of monomeric units in the alginate.

As indicated above, the present invention is useful for treating dry eye, or, more specifically, its symptoms. For that purpose, compositions for use in the present invention may be sold in a wide range of small-volume containers from 1 ml to 30 ml in size. Such containers can be made from HDPE (high density polyethylene), LDPE (low density polyethylene), polypropylene, poly(ethylene terepthalate) and the like. Flexible bottles having conventional eye-drop dispensing tops are suitable for use with the present invention.

The above-described solutions, in accordance with the present invention, may be used by instilling, for example, about one (1) or three (3) drops in the affected eye(s) as needed. The solutions are useful in one embodiment for the temporary relief of burning and irritation due to dryness in the eye and for use as a protectant against further irritation, or to relieve dryness to the eye. In another embodiment, the solutions are useful to deliver a medicament to the ocular or periocular region of a patient.

As indicated above, the present invention is useful in a contact lens cleaning, disinfecting or conditioning solution. In one embodiment, the present invention is a multipurpose cleaning solution. For that purpose, compositions for use in the present invention may be sold in a wide range of size of containers from 30 ml to 1000 ml in size. Such containers can be made from HDPE (high density polyethylene), LDPE (low density polyethylene), polypropylene, poly(ethylene terepthalate) and the like. Flexible bottles having conventional eye-drop dispensing tops are especially suitable for use with the present invention.

The above-described solutions, in accordance with the present invention, may be used by cleaning a contact lens in a solution according to one or more embodiments of the present invention. In one example, contact lenses are soaked in a volume of rapid disinfecting solution for a period that is a minimum of about 5 minutes or 10 minutes and a maximum of about two hours, about one and a half hours, about one hour. In another embodiment, the soak time may be a minimum of about one hour, about two hours, about three hours, about four hours or about six hours and/or a maximum of about 24 hours, about 12 hours, about 8 hours, about six hours, about five hours or about four hours.

The soaking may optionally be preceded and/or followed by a step of rinsing the ophthalmic solution according to one or more embodiments of the present invention for a period of about five or about ten seconds. Optionally, the contact lens can be rubbed in the hand or fingers of the user.

### EXAMPLE 1

### Formulation

The following ingredients and respective amounts are used to make a base formulation with different alginate sources having different amounts of guluronic acid residues bound to adjacent guluronic acid residues: Lessonia nigrescens (ALG-LN) and Laminaria digitata (ALG-LD).

TABLE 1
Base Alginate Formulation

| Ingredients | mg/g | % w/w |
|---|---|---|
| Boric Acid | 5 | 0.5 |
| Sodium Borate | 0.14 | 0.014 |
| Glycerin | 6 | 0.6 |
| Propylene Glycol | 6 | 0.6 |
| Alginate | 2.5 | 0.25 |
| HAP (30%) | 0.5 | 0.05 |
| Alexidine 2HCl | 3 ppm | 3 ppm |
| Purified Water | q.s. to 1000 mg | q.s. to 100% |
| | | w/w |

TABLE 2
Types of Alginate and Characterization

| Formulation | Seaweed | M/G | % M | % G | % MM | % GG |
|---|---|---|---|---|---|---|
| ALG-LD | Laminaria | 1.22 | 55 | 45 | 39 | 29 |
| | digitata | | | | | |
| ALG-LN | Lessonia | 1.50 | 60 | 40 | 43 | 23 |
| | nigrescens | | | | | |

Formulation Process: A volume of purified water that is from about 85% to about 90% of the total batch weight (the temperature of purified water should be below 40° C. before adding any raw material) is added into an appropriate stainless steel mixing vessel. Preferably, the temperature of the purified water should be below 40° C. during this step. ALG-LD and ALG-LN samples of alginate are selected. Alginate is added slowly with continued agitation and mixed thereafter for at least 45 minutes.

After the addition of Alginate and corresponding mixing, the following ingredients are slowly added in the order listed and mixed for at least 30 minutes:

### Boric Acid

### Sodium Borate

### HAP (30%)

### Glycerin

### Propylene Glycol

After these ingredients are mixed, Alexidine HCl was added via a 0.22 μm sterilizing filter and was mixed for an additional 30 minutes or more. The preparation is ready for packaging, use and storage. Refrigeration is not needed.

### EXAMPLE 2

### HPLC Analysis of Alexidine in Alginate Formulation

Solutions from alginate source (ALG-LD and ALG-LN) were prepared according to the base formulation except that zinc chloride was added in four solutions. The solutions were analyzed as follows.

A quantitative HPLC method for the determination of Alexidine dihydrochloride in the test solutions was performed. The method involved the separation of Alexidine from other formulation components using a YMC Basic reverse-phase HPLC column and a two-pump gradient system. The mobile phase gradient begins with 65% mobile phase A (acetate buffer, pH 5.1): 35% mobile phase B (acetonitrile) and ramps to 30% A: 70% B. Adjustments to the gradient composition may be made in order to optimize the chromatography.

Detection of the separated Alexidine peak is performed using UV detection at 235 nm. Quantitation of Alexidine in samples is performed versus a multi-point Alexidine standard curve generated using standards of known Alexidine dihydrochloride concentration and their respective peak area responses. The amount of Alexidine that is not bound to the alginate is disclosed. Results are displayed in Table 3.

TABLE 3
Results

| | | | Free |
|---|---|---|---|
| Sample | Zn (%) | Bottle | Alexidine |
| ALG-LN | 0.025 | PET | 2.2 |
| ALG-LN | 0.0 | PET | 2.2 |
| ALG-LD | 0.025 | PET | 0.9 |
| ALG-LD | 0.0 | PET | 0.5 |
| ALG-LN | 0.025 | LDPE | 2.3 |
| ALG-LN | 0.0 | LDPE | 2.3 |
| ALG-LD | 0.025 | LDPE | 0.9 |
| ALG-LD | 0.0 | LDPE | 1.1 |

ALG-LD has a lower amount of guluronic acid monomers bound to an adjacent guluronic acid monomer based upon the total number of monomer units in the Alginate compared to ALG-LN. ALG-LD has more than twice as much free Alexidine in solution than ALG-LN. Applicants believe that this is due to a reduction in the amount of G-G monomer pairs.

While the invention has been described in conjunction with the detailed description and specific examples, this is illustrative only. Accordingly, many alternatives, modifications and variations will be apparent to those skilled in the art in light of the foregoing description and it is, therefore, intended to embrace all such alternatives, modifications and variations as to fall within the spirit and scope of the appended claims.

What is claimed is:

1. A dry eye composition comprising an aqueous solution of alginate having a minimum of about 10% and a maximum of about 40% guluronic units bound to an adjacent guluronic unit as a percentage of the total number of monomeric units and a cationic antimicrobial agent.

2. The dry eye composition of claim 1, wherein the alginate is from Lessonia nigrescens seaweed.

3. The dry eye composition of claim 1, further comprising a polyol that is a combination of glycerin and propylene glycol.

4. The composition of claim 1, wherein the alginate has a concentration that is a minimum of about 0.01 wt. % to about 5 wt. % based upon the total weight of the solution.

5. The composition of claim 1, further comprising a polyol selected from the group consisting of glycerin, ethylene glycol, poly(ethylene glycol), propylene glycol, sorbitol, manitol and monosaccarides, disaccharides, neutral polysaccharides and oligosaccharides.

6. The composition of claim 1, wherein the cationic antimicrobial agent is selected from the group consisting of poly(hexamethylene biguanide), Alexidine, chlorhexidine, polyquaternium-1, polyquaternium-10, ammonium salts of guar, ammonium salts of chitosan, and combinations thereof.

7. The composition of claim 1, wherein the cationic antimicrobial agent is Alexidine.

8. A method of treating dry eye comprising administering to an eye a composition comprising an aqueous solution of alginate having a minimum of about 10% and a maximum of about 40% guluronic units bound to an adjacent guluronic unit as a percentage of the total number of monomeric units in the alginate and a cationic antimicrobial agent.

9. The method of claim 8, wherein the composition further comprises a polyol.

10. The method of claim 8, wherein the alginate has a concentration that is a minimum of about 0.01 wt. % and a maximum of about 5 wt. % based upon the total weight of the composition.

11. The method of claim 8, wherein the composition further comprises a polyol selected from the group consisting of glycerin, ethylene glycol, poly(ethylene glycol), propylene glycol, sorbitol, manitol and monosaccarides, disaccharides, neutral polysaccharides and oligosaccharides.

12. The method of claim 8, wherein the composition has an osmolality that is a minimum of about 200 and a maximum of about 400.

13. The method of claim 8, wherein the polyol comprises a combination of glycerin and propylene glycol.

14. The method of claim 14, wherein the cationic antimicrobial agent is selected from the group consisting of poly(hexamethylene biguanide), Alexidine, chlorhexidine, polyquatemium-1, polyquaternium-10, quaternary ammonium salts of guar, quaternary ammonium salts of chitosan, and combinations thereof.

15. The method of claim 18, wherein the cationic antimicrobial agent is Alexidine.

16. The method of claim 8, wherein the cationic antimicrobial agent is polyquaternium-1 or polyquaternium-10.

17. A method of manufacturing an ophthalmic composition comprising combining in an aqueous solution ophthalmically pure alginate having a minimum of about 0% and a maximum of about 50% guluronic units bound to an adjacent guluronic unit as a percentage of the total number of monomeric units, and a cationic antimicrobial agent.
